(19) United States
(12) Patent Application Publication
(10) Pub. No.: US 2020/0055088 A1
(43) Pub. Date: Feb. 20, 2020

(54) ULTRASONIC SENSOR

(71) Applicant: KYOCERA Corporation, Kyoto-shi, Kyoto (JP)

(72) Inventor: Hiroki OKADA, Machida-shi (JP)

(73) Assignee: KYOCERA Corporation, Kyoto-shi, Kyoto (JP)

(21) Appl. No.: 16/487,875

(22) PCT Filed: Feb. 14, 2018
(86) PCT No.: PCT/JP2018/005036

§ 371 Date: Aug. 22, 2019

(30) Foreign Application Priority Data
Feb. 24, 2017 (JP) 2017-033433

(51) Int. Cl. B06B 1/06; H01L 41/113; H01L 41/187; G01H 11/08; A61B 8/00

(52) U.S. Cl. CPC B06B 1/0666; H01L 41/1132; A61B 8/4483; H01L 41/1876; G01H 11/08; H01L 41/1138

(57) ABSTRACT

A sensor includes a lower electrode layer facing a cavity, a piezoelectric layer located on the lower electrode layer, and an upper electrode layer located on the piezoelectric layer. The piezoelectric layer includes a first piezoelectric part and a second piezoelectric part. The first piezoelectric part includes a first material having piezoelectricity and at least partially overlays the cavity when viewed on a plane. The second piezoelectric part includes a second material having piezoelectricity and being different in at least one of a “g” constant and “d” constant from the first material and, when viewed on a plane, is located in a region different from an arrangement region of the first piezoelectric part and at least partially overlays the cavity.

### TECHNICAL FIELD

The present disclosure relates to a pMUT (piezoelectric micromachined ultrasonic transducer) or another piezoelectric type ultrasonic sensor.

### BACKGROUND ART

As an ultrasonic sensor utilized for an ultrasonic probe of an ultrasonic diagnosis device etc., there is known one using a piezoelectric film (Patent Literatures 1 and 2). For example, an ultrasonic sensor has a membrane, lower electrode, piezoelectric film, and upper electrode laid in that order above a cavity. In the piezoelectric film, the thickness direction is made the polarization direction (direction of spontaneous polarization).

When voltage is applied to a piezoelectric film in its thickness direction, the piezoelectric film expands and contracts in its planar direction. This expansion and contraction is restricted by the membrane. Accordingly, the multilayer member including the membrane and piezoelectric film flexurally deforms in the overlaid direction like a bimetal. In turn, a pressure wave is formed in the atmosphere around the multilayer member. Further, when electrical signals changing in voltages with suitable waveforms are input to a lower electrode and upper electrode, ultrasound reflecting the waveforms of those electrical signals (for example reflecting the frequencies) is transmitted. Further, by an action reverse to that described above, the ultrasound received by the multilayer member is converted to an electrical signal reflecting the waveform of that ultrasound.

Patent Literature 1 discloses an ultrasonic sensor in which a reception-use piezoelectric film and a transmission-use piezoelectric film made of a material different from that for the former piezoelectric film are overlaid on a membrane. Patent Literature 2 discloses an ultrasonic sensor which, when viewed on a plane, has a piezoelectric film positioned at the center of a cavity and a piezoelectric film which is made of the same material as that for the former piezoelectric film and is positioned at an outer periphery of the cavity.

### CITATION LIST

### Patent Literature

Patent Literature 1: Japanese Patent Publication No. 2013-146478

Patent Literature 2: Japanese Patent Publication No. 2013-135793

### SUMMARY OF INVENTION

An ultrasonic sensor according to one aspect of the present disclosure includes a lower electrode layer which faces a cavity, a piezoelectric layer which is located on the lower electrode layer, and an upper electrode layer which is located on the piezoelectric layer. The piezoelectric layer includes a first piezoelectric part and a second piezoelectric part. The first piezoelectric part is formed by a first material having piezoelectricity and at least partially overlays the cavity when viewed on a plane. The second piezoelectric part is formed by a second material having piezoelectricity and being different in at least any one of a “g” constant or “d” constant from the first material and, when viewed on a plane, is located in a region different from an arrangement region of the first piezoelectric part and at least partially overlays or is adjacent to the cavity.

### BRIEF DESCRIPTION OF DRAWINGS

FIG. 1A is a cross-sectional view showing a configuration of an ultrasonic sensor according to an embodiment, and FIG. 1B is a plan view of the ultrasonic sensor in FIG. 1A.

FIG. 2A and FIG. 2B are schematic cross-sectional views showing one example and another example of the materials for a piezoelectric part, and FIG. 2C and FIG. 2D are schematic cross-sectional views for explaining the action of the ultrasonic sensor according to the embodiment.

FIG. 3A is a schematic view showing one example of the configurations of a transmission part and reception part, while FIG. 3B is a schematic view showing another example of the configurations of the transmission part and reception part.

FIG. 4A is a plan view showing the configuration of a sensor according to one modification, while FIG. 4B is a plan view showing the configuration of a sensor according to another modification.

FIG. 5A and FIG. 5B are schematic cross-sectional views showing one modification and another modification relating to the thickness of the piezoelectric layer, while FIG. 5C, FIG. 5D, and FIG. 5E are views schematically showing one modification, another modification, and still another modification relating to the planar shapes of the upper electrode layer etc.

FIG. 6 is a block diagram schematically showing the configuration of an ultrasonic diagnosis device as an example of application of the ultrasonic sensor.

### DESCRIPTION OF EMBODIMENTS

Below, embodiments according to the present disclosure will be explained with reference to the drawings. Note that, the following drawings are schematic ones. Therefore, details will be sometimes omitted. Further, size ratios etc. do not always coincide with the actual ones. Further, size ratios among the plurality of drawings do not always coincide with each other.

The drawings, for convenience, will sometimes have an orthogonal coordinate system D1-D2-D3 attached. Note that, in the sensor, any direction may be defined as “above” or “below”. In the explanation of the embodiments, however, sometimes the “upper part” or “lower part” and other terms will be used where the positive side of the D3 axis direction is the upper part. Further, when referred to as “viewed on a plane” in the following description, it means “viewed in the D3 axis direction” unless particularly explained otherwise.

In the explanation of the present embodiments, sometimes the names of the materials will be mentioned. The “names of the materials” will designate the principal ingredients of the materials. The materials may suitably contain additives. The “principal ingredients” are for example ingredients with ratios of atoms relative to all of the atoms in that material exceeding 50%.

(Overall Configuration of Sensor)

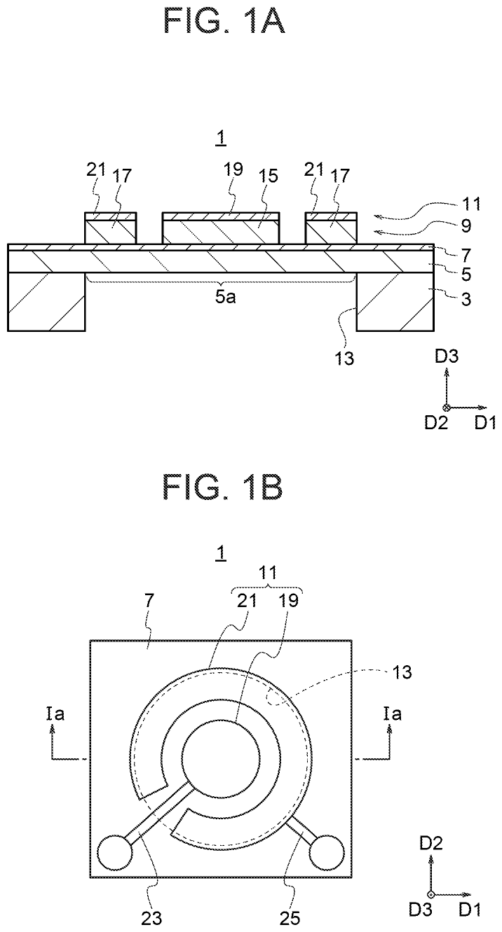

FIG. 1A is a cross-sectional view showing the configuration of an ultrasonic type sensor 1 according to an embodiment, and FIG. 1B is a plan view showing the configuration of the sensor 1. Note that, FIG. 1A corresponds to the Ia-Ia line in FIG. 1B.

The sensor 1 is for example configured as a pMUT. The sensor 1 for example receives as input an electrical signal changing in voltage by a predetermined waveform (for example rectangular wave or sine wave). Further, the sensor 1 converts that electrical signal to ultrasound reflecting the waveform of the electrical signal (for example reflecting the frequency) and transmits the result to one of the positive side and negative side in the D3 axis direction. Further, for example, the sensor 1 receives ultrasound from the one of the positive side and negative side in the D3 axis direction and converts the ultrasound to an electrical signal reflecting the waveform of the ultrasound.

Note that, the positive side or negative side in the D3 axis direction for transmission and reception of the ultrasound referred to here is not always parallel to the D3 axis direction. Further, the frequency band of the ultrasound is for example a frequency band of 20 kHz or more. The upper limit of the frequency of the ultrasound is not particularly restricted. However, for example, the upper limit is 5 GHz.

The sensor 1, for example, as shown in FIG. 1A, has a base body 3, membrane 5, lower electrode layer 7, piezoelectric layer 9, and upper electrode layer 11 which are overlaid in that order from the lower part.

The base body 3 for example has a cavity 13. The various members (5, 7, 9, 11, etc.) above the base body 3 configure a vibration region part vibrating for transmission and reception of ultrasound by the portions positioned above the cavity 13. The vibration region part, for vibration of a primary mode of flexural deformation which will be explained later, may be configured so that the resonance frequency is positioned in the frequency band of the ultrasound.

The cavity 13 may be a concave shaped one which opens upward in the base body 3 or a via hole-shaped one penetrating through the base body 3. The planar shape and dimensions of the cavity 13 may be suitably set. In the example shown, the planar shape of the cavity 13 is circular. Further, the planar shape is constant in the depth direction (D3 axis direction) of the cavity 13.

Note that, unlike the example shown, the planar shape of the cavity 13 need not be constant in the depth direction of the cavity 13. For example, the cavity 13 may become smaller in diameter toward the upper surface side as well. When considering such an aspect, in the explanation of the present embodiment etc., the term of the “planar shape of the cavity 13” may be grasped as designating the planar shape of the upper surface of the cavity 13. This is because, mainly, it is the upper surface of the cavity 13 that restricts the region of the membrane 5 etc. which can vibrate.

The material of the base body 3 may be any material. Further, the base body 3 may be integrally formed or may be formed by a combination of a plurality of members. For example, the material for the base body 3 is an inorganic insulating material or organic insulating material. More specifically, for example, the base body 3 may be integrally formed by silicon (Si) or another insulating material. Further, for example, the base body 3 may be integrally formed in substantially its entirety by silicon or another insulating material and may have a layer made of SiO₂ or another insulating material on its upper surface.

The membrane 5 is for example layer shaped having a constant thickness and covers the cavity 13. The area of the membrane 5 is broader than the area of the cavity 13. The membrane 5 is fixed to the base body 3 on the periphery of the cavity 13 to be supported there. Note that, in the membrane 5, a region overlaying the cavity 13 will be sometimes referred to as a “vibration part 5a”. The thickness of the membrane 5 may be suitably set.

The membrane 5 is for example formed by an insulating material. The insulating material may be an inorganic material or organic material. More specifically, for example, it is silicon, silicon oxide (SiO₂), or silicon nitride (SiN). Note that, the membrane 5 may be configured by overlaying a plurality of layers which are made of different materials from each other as well. Further, the membrane 5 may be made of the same material as that for the base body 3 and integrally formed with the base body 3. Further, the membrane 5, unlike the example shown, may be formed by a conductive material and may configure part or all of the lower electrode layer (may function also as the lower electrode layer).

The lower electrode layer 7 is for example layer shaped having a constant thickness. It forms a solid pattern electrode having a size crossing over the inside and outside of the cavity 13 when viewed on a plane. However, the lower electrode layer 7 for example may also be configured by only a region overlaying the cavity 13 or may be configured by only a region which the piezoelectric layer 9 overlays. The thickness of the lower electrode layer 7 may be suitably set. The material for the lower electrode layer 7 may be made a suitable metal. For example, it is gold (Au), platinum (Pt), aluminum (Al), copper (Cu), or chromium (Cr). The lower electrode layer 7 may be configured by overlaying a plurality of layers which are formed by mutually different materials as well.

The piezoelectric layer 9 has a first piezoelectric part 15 formed by a first material and a second piezoelectric part 17 formed by a second material different from the first material. The first material and second material will be explained later. The thicknesses of the first piezoelectric part 15 and second piezoelectric part 17 are for example the same as each other. The planar shapes of the first piezoelectric part 15 and second piezoelectric part 17 are for example the same as the later explained planar shapes of the first electrode part 19 and second electrode part 21 which are provided in the upper electrode layer 11. In the following explanation of the planar shapes of the first piezoelectric part 15 and second piezoelectric part 17, the planar shapes of the first electrode part 19 and second electrode part 21 in FIG. 1B may be referred to.

The first piezoelectric part 15, for example, includes a part positioned at the center of the cavity 13 when viewed on a plane. Note that, “the center of the cavity 13 when viewed on a plane” is for example the center of gravity of the figure. The center of gravity of the figure is the point where the primary moment around that is 0. In the example shown, the planar shape of the cavity 13 is circular, therefore the center of gravity of the figure is the center of the circle. Further, the first piezoelectric part 15, when viewed on a plane, is smaller than the cavity 13 and falls within the cavity 13.

The planar shape of the first piezoelectric part 15 is for example made substantially the same as the planar shape of the cavity 13 and/or has an outer edge having substantially a constant distance from the outer edge of the cavity 13. In the example shown, the planar shape of the cavity 13 is circular, therefore the first piezoelectric part 15 is circular shaped concentrically with the cavity 13 and has a smaller diameter than the cavity 13.

The second piezoelectric part 17, for example, when viewed on a plane, includes a portion which is positioned at outer side of the cavity 13 from the first piezoelectric part 15. More specifically, for example, the second piezoelectric part 17 has a shape that surrounds the first piezoelectric part 15 when viewed on a plane. When referring to “surrounds the first piezoelectric part 15”, for example, when considering the range of angle around the center of gravity of the figure of the first piezoelectric part 15, the sum of the range of angle in which the second piezoelectric part 17 exists only have to exceed 180° and/or the largest range of angle in which the second piezoelectric part 17 does not exist only have to be less than 120°.

In the example shown, the second piezoelectric part 17 extends over a range exceeding a semicircle (180°) so as to surround the first piezoelectric part 15, and satisfies both of the two conditions described above. More specifically, for example, the second piezoelectric part 17 extends over a range exceeding 270° with a constant width. The shape thereof is made substantially the same as the outer edge of the first piezoelectric part 15 (and/or cavity 13) and/or has an inner edge and outer edge having substantially constant distances from the outer edge of the first piezoelectric part 15 (and/or cavity 13). In the example shown, the shape of the second piezoelectric part 17 is an arc shape.

The second piezoelectric part 17 for example substantially falls within the cavity 13 when viewed on a plane. In other words, the second piezoelectric part 17 has a region overlaying the cavity 13 when viewed on a plane and the area of the overlaying region is larger than the area (substantially 0 in the example shown) of the region which does not overlay it. Further, from another viewpoint, in the example shown, the outer edge of second piezoelectric part 17 substantially matches the outer edge of the cavity 13.

The upper electrode layer 11 for example has a first electrode part 19 positioned on the first piezoelectric part 15 and a second electrode part 21 positioned on the second piezoelectric part 17. As explained above, in the present embodiment, the planar shapes of the first electrode part 19 and second electrode part 21 are substantially the same as the planar shapes of the first piezoelectric part 15 and second piezoelectric part 17, therefore the explanation relating to the planar shapes of the first piezoelectric part 15 and second piezoelectric part 17 may be applied as is to the explanation of the planar shapes of the first electrode part 19 and second electrode part 21 by replacing the “first piezoelectric part 15” and “second piezoelectric part 17” by the “first electrode part 19” and “second electrode part 21”.

The materials and thicknesses of the first electrode 19 and the second electrode part 21 are for example the same as each other. However, these materials and/or thicknesses may be different from each other as well. Each electrode part is for example layer shaped having a constant thickness. Its thickness may be suitably set. The material for the upper electrode layer 11 may be the same as or different from the material for the lower electrode layer 7. The specific materials thereof may be for example the materials mentioned in the explanation of the materials for the lower electrode layer 7. The upper electrode layer 11 may be configured by superposing a plurality of layers which are formed by mutually different materials as well.

The sensor 1 may have suitable connection conductors as well for input of signals (voltages) to the lower electrode layer 7 and upper electrode layer 11 and output of the signals (for example voltages) from these electrode layers. In FIG. 1B, a first connection conductor 23 led out of the first electrode part 19 and a second connection conductor 25 led out of the second electrode part 21 are illustrated.

The first connection conductor 23 and second connection conductor 25 are for example configured by layer shaped conductors which are provided on not shown insulation layers positioned on the lower electrode layer 7. These not shown insulation layers for example have substantially the same planar shapes as those of the first connection conductor 23 and second connection conductor 25. In FIG. 1B, they are hidden behind the first connection conductor 23 and second connection conductor 25, so are not shown.

Note that, the not shown insulation layer may be provided in solid pattern at a position where the first piezoelectric part 15 and second piezoelectric part 17 are not arranged. Further, the thicknesses of these not shown insulation layers may be equal to the thickness of the piezoelectric layer 9, may be thinner, or may be thicker. When the thicknesses are equal, these not shown insulation layers may be integrally formed with one of the first piezoelectric part 15 and second piezoelectric part 17 by the same material.

Further, for example, the insulation layers described above need not be provided either. For example, the regions overlaying the first connection conductor 23 and second connection conductor 25 may be made regions where the lower electrode layer 7 is not arranged. Further, the first connection conductor 23 and second connection conductor 25 may be configured by for example layer shaped conductors which are directly provided on the membrane 5.

The front ends of the first connection conductor 23 and second connection conductor 25 are for example connected to not shown via conductors passing through the membrane 5 or the membrane 5 and at least parts of the base body 3. Further, the lower electrode layer 7 is connected to the via conductors as described above at suitable positions. Further, input and output of signals are carried out with the lower electrode layer 7 and upper electrode layer 11 through the via conductors described above. Note that, in place of the via conductors, pads etc. of a flexible board may be joined from the upper surface side of the sensor 1 to the first connection conductor 23, the second connection conductor 25, and a suitable portion in the lower electrode layer 7 as well.

In the example in FIG. 1B, the first electrode part 19 and first connection conductor 23 and the second electrode part 21 and second connection conductor 25 are not connected. Accordingly, the signals can be input and output with respect to the first electrode part 19 and the second electrode part 21 (from another viewpoint, the first piezoelectric part 15 and the second piezoelectric part 17) separately from each other.

(Material for Piezoelectric Layer)

The first material configuring the first piezoelectric part 15 and the second material configuring the second piezoelectric part 17 are piezoelectric materials. As piezoelectric materials, for example, there can be mentioned aluminum nitride (AlN), barium titanate (BTO: BaTiO₃), potassium sodium niobite (KNN: (K,Na)NbO₃), sodium bismuth titanate (NBT: Na_{0.5}Bi_{0.5}TiO₃), and lead zirconate titanate (PZT: Pb(Zrₓ,Ti₁₋ₓ)O₃). As will be understood also from the above illustration, the piezoelectric materials may be or may not be ferroelectric materials and may be or may not be pyroelectric materials. Further, the crystal structures may be suitable ones such as the perovskite type or wurtzite type.

Values of −d₃₁ and g₃₁ in the “d” constant (piezoelectric distortion constant) and “g” constant (piezoelectric output constant) of the piezoelectric materials illustrated in the above description will be shown below. If described for confirmation, the suffixes 1 and 3 indicate the axial directions by tensor notation. The triaxial direction is the polarization direction (D3 axis direction), and the uniaxial direction is the direction (D1 axis direction) perpendicular to the polarization direction. Note that, even if the names (principal ingredients) are the same, the values of the “g” constant and “d” constant will differ due to the differences of additives and manufacturing methods. Therefore, in the following description, the values of −d₃₁ and g₃₁ will be shown in terms of range.

| | −d₃₁ (pm/V) | g₃₁ (10⁻² V · N/m) |
|---|---|---|
| ALN | 8 to 14 | 7.8 to 8.5 |
| BTO | 65 | 2.4 |
| KNN | 80 to 138 | 2.3 to 3.9 |
| NBT | 40 to 206 | 0.7 to 2.4 |
| PZT | 132 to 220 | 1.2 to 2.4 |

As already explained, the first material configuring the first piezoelectric part 15 and the second material configuring the second piezoelectric part 17 are materials which are different from each other. “Different” referred to here for example means that at least one of the “g” constant and the “d” constant is different. The names and/or principal ingredients may be the same. Further, as will be understood from the action which will be explained later, in the piezoelectric layer 9, the thickness direction (D3 axis direction) is made the polarization direction. The voltage or electrical displacement in the polarization direction and a strain or stress in the planar direction (D1 axis direction and D2 axis direction) are basically utilized. Accordingly, in the judgment of whether the first material and the second material are the same, the “g” constant and/or “d” constant may be represented by −d₃₁ and/or g₃ᵢ.

One of the first material and second material is for example larger in the “d” constant and smaller in the “g” constant relative to the other. The combination of such two types of materials may be suitably set. For example, use will be made of the materials described above illustrating the values of −d₃₁ and/or g₃₁. In this case, for example, when the other material (material having a relatively larger “g” constant) is AlN, the one material (material having a relatively larger “d” constant) is BTO, KNN, NBT, or PZT. Further, for example, when the other material is BTO, KNN can be used as the one material.

(Combination of Positions and Materials of Piezoelectric Parts)

In the following explanation, as described above, an aspect where one of the first material and second material is larger in the “d” constant and smaller in the “g” constant compared with the other will be used as an example. Further, as the combination of the one material and the other material, PZT and AlN will be used as an example.

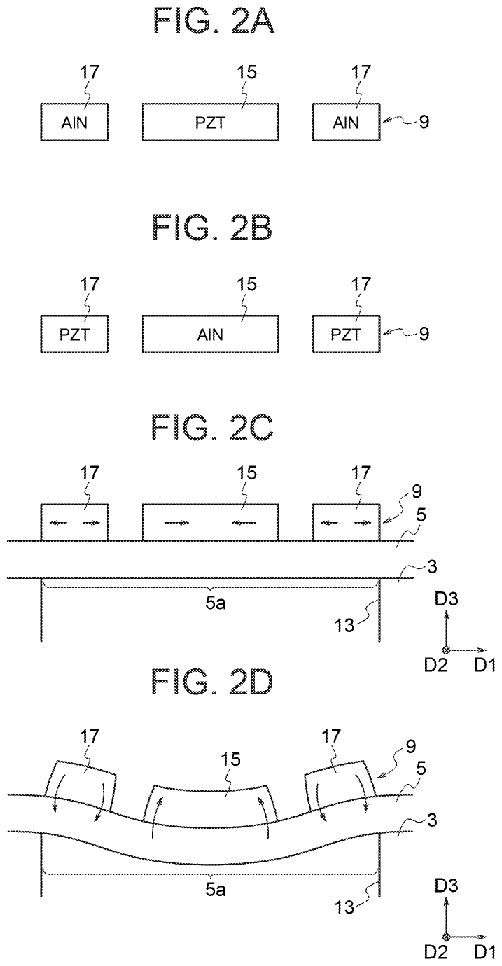

FIG. 2A is a schematic cross-sectional view (hatching showing the cross-section is omitted) of the piezoelectric layer 9 for showing the example of materials of the first piezoelectric part 15 and second piezoelectric part 17. In this example, the first material (PZT) of the first piezoelectric part 15 is made a material that is larger in the “d” constant and smaller in the “g” constant compared with the second material (AlN) of the second piezoelectric part 17.

FIG. 2B is a schematic cross-sectional view of the piezoelectric layer 9 for showing another example of materials of the first piezoelectric part 15 and second piezoelectric part 17. In this example, conversely to FIG. 2A, the first material (AlN) of the first piezoelectric part 15 is made a material that is smaller in the “d” constant and larger in the “g” constant compared with the second material (PZT) of the second piezoelectric part 17.

In this way, either of the first piezoelectric part 15 or second piezoelectric part 17 may be made relatively larger in the “d” constant and relatively smaller in the “g” constant.

(Action of Sensor)

FIG. 2C and FIG. 2D are schematic cross-sectional views (hatchings showing cross-sections are omitted) for explaining the action of the sensor. In these views, illustration of the lower electrode layer 7 and upper electrode layer 11 is omitted.

In both of the first piezoelectric part 15 and second piezoelectric part 17, the thickness direction (D3 axis direction) is made the polarization direction. Further, in FIG. 2C, the first piezoelectric part 15 is supplied with a voltage by the same direction as the direction of polarization. Therefore, as indicated by arrows, the part contracts in the planar direction (D1 axis direction and D2 axis direction). Conversely, the second piezoelectric part 17 is supplied with voltage by an inverse direction to the direction of polarization. Therefore, as indicated by arrows, the part extends in the planar direction.

Accordingly, as indicated by arrows in FIG. 2D, the region in the membrane 5 (vibration part 5a) overlaying the first piezoelectric part 15 and the first piezoelectric part 15 warp to the membrane 5 side (cavity 13 side) like a bimetal. On the other hand, the region in the membrane 5 (vibration part 5a) overlaying the second piezoelectric part 17 and the second piezoelectric part 17 warp to the inverse side to the membrane 5 (cavity 13) like a bimetal. Further, the entireties of the vibration part 5a and piezoelectric layer 9 displace to the cavity 13 side.

In the above description, the explanation was given by taking as an example the case where the vibration part 5a warps to the cavity 13 side. Conversely to the above description, if voltage is supplied to the first piezoelectric part 15 by an inverse direction to the direction of polarization, and voltage is supplied to the second piezoelectric part 17 by the same direction as the direction of polarization, according to the same principle, the vibration part 5a displaces to the opposite side from the cavity 13.

Due to the displacement of the vibration part 5a as described above, a pressure wave is formed in the atmosphere around the vibration part 5a. Further, due to electrical signals changing in voltages with predetermined waveforms being input to the lower electrode layer 7 and upper electrode layer 11, an ultrasound reflecting the waveforms (for example frequencies) of the electrical signals is generated.

Note that, the electrical signal for example may be one by which application of voltage making the vibration part 5a displace to the cavity 13 side and application of voltage making the vibration part 5a displace to the side opposite to the cavity 13 are repeated. That is, the electrical signal may be one by which the polarity (positive and negative) is inverted (the directions of the voltages (electric fields) are alternately replaced by each other in the D3 axis direction).

Further, for example, the electrical signal may be one by which only application of voltage making the vibration part 5a displace to the cavity 13 side or only application of voltage making the vibration part 5a displace to the side opposite to the cavity 13 is repeated. In this case, the ultrasound is generated by repetition of warping and cancellation of warping by a restoring force.

Further, the waveform of the electrical signal may be a suitable one. For example, in one ultrasonic signal (echo signal), the number of waves may be suitably set, and the frequency and voltage may be constant or may not be constant. In the case where the polarity of the electrical signal changes, the voltage on the positive side and the voltage on the negative side may have the same magnitude or have different magnitudes.

The transmission of the ultrasound was explained. The reception of the ultrasonic is realized by an inverse principle to that at the time of transmission. The sensor 1 for example intermittently transmits the ultrasonic signal and receives the ultrasonic signal in the periods where the ultrasonic signal is not transmitted. Due to this, the sensor 1 for example receives the ultrasonic signal which was transmitted by itself, reflected, and returned.

(Example of Configurations of Transmission Part and Reception Part)

### Configuration Example 1

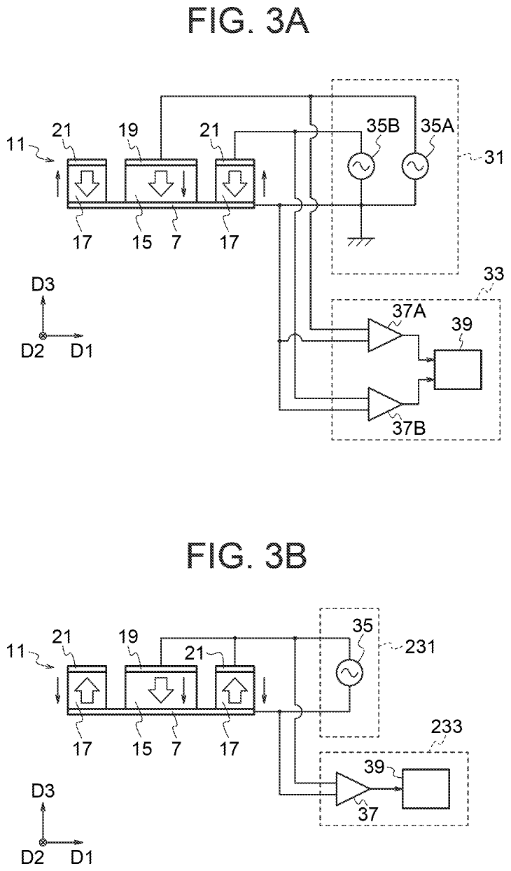

FIG. 3A is a schematic view showing an example of the configurations of a transmission part 31 outputting an electrical signal to the sensor 1 and a reception part 33 receiving as input an electrical signal from the sensor 1.

In this example, the first piezoelectric part 15 and the second piezoelectric part 17, as indicated by white arrows, are made the same in the directions of polarization as each other. Note that, in the example shown, the direction of polarization is downward. However, naturally it may be upward as well. Further, as indicated by the black arrows, in the transmission part 31, voltages are supplied to the first piezoelectric part 15 and second piezoelectric part 17 so that the directions of the voltages supplied in the thickness direction (D3 axis direction) become inverse directions to each other between these piezoelectric parts. Due to this, as explained with reference to FIG. 2C and FIG. 2D, one of the first piezoelectric part 15 and second piezoelectric part 17 is supplied with voltage having the same direction as the direction of polarization and contracts in the planar direction, and the other is supplied with voltage in the inverse direction to the direction of polarization and extends in the planar direction.

Specifically, for example, the transmission part 31 has a first driving part 35A connected to the first electrode part 19 and a second driving part 35B connected to the second electrode part 21 (sometimes they will be simply referred to as the “driving parts 35”). These driving parts 35, as indicated by notations showing power supplies for convenience, for example, are configured including power supply circuits converting a commercial power supply to signals of voltages having suitable waveforms and outputting the results. Further, the lower electrode layer 7, for example, is given a reference potential. Accordingly, by application of voltages having mutually inverse polarities (positive and negative relative to the reference potential) to the first electrode part 19 and second electrode part 21 by the two driving parts 35, voltages having mutually inverse directions relative to the thickness directions can be supplied to the first piezoelectric part 15 and second piezoelectric part 17.

Note that, the positive and negative sign of the potential of the first electrode part 19 or second electrode part 21 relative to the potential of the lower electrode layer 7 (positive and negative sign relative to the reference potential) may change or not change as understood from the explanation given with reference to FIG. 2C and FIG. 2D. Further, in a case where it does not change, any one of a positive or negative sign may be utilized. Further, the two types of signals output by the two driving parts 35, except for the fact that the polarities are inverse, may be signals having the same waveforms as each other or may be signals having mutually different waveforms. For example, the magnitudes of the voltages are different or the phases are somewhat deviated. Further, by one driving part 35, potentials having inverse polarities to each other may be given to the first electrode part 19 and second electrode part 21 while connecting the lower electrode layer 7 and the reference potential part as well.

The reception part 33, according to the inverse principle to that for the transmission part 31, can receive electrical signals having inverse polarities to each other from the first electrode part 19 and second electrode part 21. Specifically, for example, the reception part 33 has a first detection part 37A which detects the voltage between the lower electrode layer 7 and the first electrode part 19 and a second detection part 37B which detects the voltage between the lower electrode layer 7 and the second electrode part 21 (sometimes they will be simply referred to as the “detection parts 37”). These detection parts 37, as indicated by notations showing amplifiers for convenience and, for example, are configured including voltage amplifiers amplifying the input voltages and outputting the results. Further, the reception part 33, for example, has a processing part 39 which makes the polarities of the signals amplified by the two detection parts 37 the same and then adds the results.

Note that, the potentials of the first electrode part 19 and the second electrode part 21 are inverse in polarity, therefore the potential difference of the two may be detected by one detection part 37 connected to them as well. Further, the amplifiers included in the first detection part 37A and second detection part 37B may be formed as charge amplifiers in place of voltage amplifiers as well.

### Example of Configuration 2

FIG. 3B is a schematic view showing another example of the configurations of the transmission part and reception part.

In this example, in the first piezoelectric part 15 and second piezoelectric part 17, the directions of polarization are made inverse to each other as indicated by the white arrows. Note that, in the example shown, the direction of polarization in the first piezoelectric part 15 is made downward, and the direction of polarization in the second piezoelectric part 17 is made upward. However, conversely, naturally the direction may be upward in the first piezoelectric part 15 and downward in the second piezoelectric part 17. Further, as indicated by the black arrows, a transmission part 231 supplies voltages to the first piezoelectric part 15 and second piezoelectric part 17 so that the directions of voltage supplied in the thickness direction (D3 axis direction) become the same directions as each other between these piezoelectric parts. The first piezoelectric part 15 and the second piezoelectric part 17 are mutually inverse in the directions of polarization, therefore the relative relationships between the directions of polarization and directions of voltage become inverse between the first piezoelectric part 15 and the second piezoelectric part 17. Due to this, as explained with reference to FIG. 2C and FIG. 2D, one of the first piezoelectric part 15 and second piezoelectric part 17 extends in the planar direction while the other contracts in the planar direction.

Specifically, for example, the first electrode part 19 and the second electrode part 21 are connected to each other. The transmission part 231 has a driving part 35 which is connected to these mutually connected first electrode part 19 and second electrode part 21 (upper electrode layer 11). The configuration of the driving part 35 is the same as that in FIG. 3A. Further, the driving part 35 supplies voltage to between the upper electrode layer 11 (19 and 21) and the lower electrode layer 7.

Note that, positive and negative polarity of the potential of the first electrode part 19 and second electrode part 21 relative to the potential of the lower electrode layer 7 may change or may not change in the same way as FIG. 3A. Further, when it does not change, either of the positive or negative polarity may be utilized. Further, either of the upper electrode layer 11 and lower electrode layer 7 may be given the reference potential as well (potential need not change either).

A reception part 233, according to the inverse principle to that of the transmission part 231, can receive polarities which are the same as each other from the first electrode part 19 and the second electrode part 21. Specifically, for example, the reception part 233 has a detection part 37 which detects voltage between the lower electrode layer 7 and the upper electrode layer 11 (mutually connected first electrode part 19 and second electrode part 21). The configuration of the detection part 37 is the same as that in FIG. 3A.

Note that, the transmission part 231, in the same way as the transmission part 31 in FIG. 3A, may have two driving parts 35 corresponding to the first electrode part 19 and second electrode part 21 as well. In this case, for example, the two driving parts 35 can output signals having mutually different waveforms to the first electrode part 19 and second electrode part 21, for example, signals which are different in magnitudes of voltages or somewhat offset in phases.

(Method for Manufacturing Sensor)

The method for manufacturing the sensor may be made the same as various known manufacturing methods except that mutually different materials are used for the first piezoelectric part 15 and the second piezoelectric part 17. For example, a wafer for forming the base 3 may be repeatedly formed with a thin film and patterned to thereby to form the membrane 5, lower electrode layer 7, piezoelectric layer 9, and upper electrode layer 11. At this time, the first piezoelectric part 15 and the second piezoelectric part 17 may be formed at different processes of the thin film formation and patterning unlike the conventional case.

Note that, the formation of thin film and the patterning may be separately carried out or may be simultaneously carried out by formation of the thin film through a mask. Further, various known methods may be used for the method of formation of the thin film for forming the first piezoelectric part 15 and second piezoelectric part 17. For example, the thin film of the piezoelectric material may be formed by sputtering or may be formed by formation of a film of a sol-like or gel-like piezoelectric material and heat treatment. The polarization direction may be set by control of the orientation of the crystal etc. at the time of formation of the film of the piezoelectric material. Otherwise, if the piezoelectric material is a ferroelectric material, it may be set by polarization after formation of the film as well.

As described above, in the present embodiment, the sensor 1 has a membrane 5 having a vibration part 5a covering a cavity 13, a lower electrode layer 7 overlaying the vibration part 5a, a piezoelectric layer 9 overlaying the lower electrode layer 7 on the side opposite to the vibration part 5a, and an upper electrode layer 11 overlaying the piezoelectric layer 9 on the side opposite to the lower electrode layer 7. The piezoelectric layer 9 has a first piezoelectric part 15 and a second piezoelectric part 17. The first piezoelectric part 15 includes a first material having piezoelectricity and at least partially overlays the vibration part 5a when viewed on a plane. The second piezoelectric part 17 includes a second material having piezoelectricity and being different in at least one of a “g” constant and “d” constant from the first material and, when viewed on a plane, is positioned in a region different from an arrangement region of the first piezoelectric part 15 and at least partially overlays the vibration part 5a.

Accordingly, for example, by combining the first piezoelectric part 15 which includes the first material suitable for one of the transmission and reception and the second piezoelectric part 17 which includes the second material suitable for the other of the transmission and reception, the transmission strength and the reception sensitivity can be improved. Further, for example, compared with the case where the first piezoelectric part 15 and the second piezoelectric part 17 are overlaid, the layer including the membrane 5 and piezoelectric layer 9 is thin, therefore this layer can be improved in flexibility. Further, for example, in the piezoelectric layer 9, when the vibration part 5a displaces to one side in the thickness direction, there are a region where it contracts and a region where it expands. The magnitudes of stress at them differ from each other. Therefore, it is also possible to improve the transmission strength and/or reception sensitivity by selecting a suitable material in accordance with the region.

In the present embodiment, one material (for example PZT) of the first material and second material is larger in the “d” constant and smaller in the “g” constant relative to the other material (for example (AlN)).

Accordingly, for example, the transmission strength can be improved by the material having the relatively larger “d” constant while the reception sensitivity can be improved by the material having the relatively larger “g” constant.

In the present embodiment, the first piezoelectric part 15 includes a part which is positioned at the center of the vibration part 5a when viewed on a plane. The second piezoelectric part 17 includes a part which is positioned on the outer side of the vibration part 5a from the first piezoelectric part 15 when viewed on a plane.

Accordingly, for example, when the vibration part 5a displaces to one side of the thickness direction thereof, one of the first piezoelectric part 15 and second piezoelectric part 17 includes a region which expands, and the other includes a region which contracts. By arranging mutually different materials in regions different in the mode of deformation from each other, the transmission strength and/or reception sensitivity is improved.

For example, as described above, in the case where the first piezoelectric part 15 includes a part positioned at the center of the vibration part 5a, as illustrated in FIG. 2A, the first material is made larger in the “d” constant than the second material, and the second material is made larger in the “g” constant than the first material.

In this case, for example, by using a material having a relatively larger “d” constant for the first piezoelectric part 15 positioned at the center side which has a great influence upon the transmission, the transmission strength can be improved. On the other hand, in reception, the stress outside of the vibration part 5a is apt to become large. Accordingly, by using a material having a large “g” constant for the second piezoelectric part 17 positioned on the outside in this way, the reception sensitivity can be improved.

Alternatively, for example, in the case as described above where the first piezoelectric part 15 includes a part positioned at the center of the vibration part 5a, as illustrated in FIG. 2B, the first material is made larger in the “g” constant than the second material, and the second material is made larger in the “d” constant than the first material.

In this case, for example, a material having a larger “d” constant is used for the second piezoelectric part 17 positioned on the outside where the stress is apt to become larger at the time of reception, therefore the charges generated along with the reception are apt to become larger. Accordingly, for example, in the case where use is made of a charge amplifier as the amplifier configuring the detection part 37, the reception sensitivity can be improved.

In the present embodiment, the first piezoelectric part 15 includes a part positioned at the center of the vibration part 5a, and the second piezoelectric part 17 extends over a range exceeding a semicircle so as to surround the first piezoelectric part 15 when viewed on a plane.

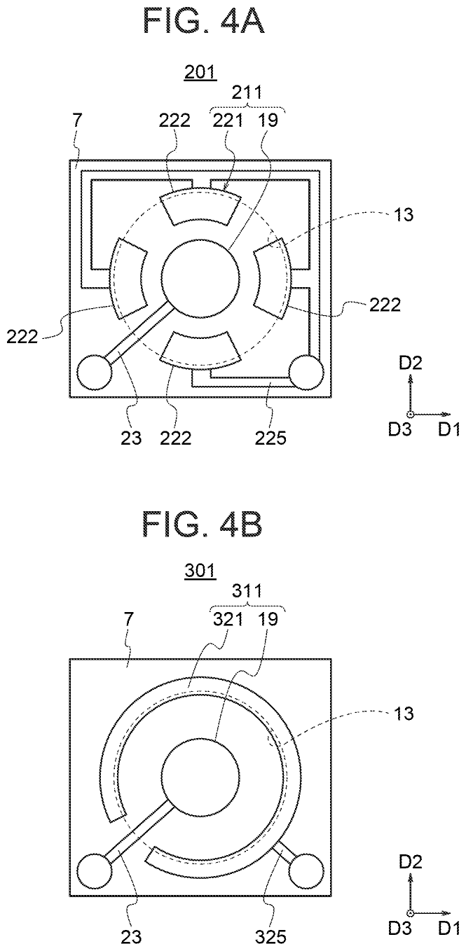

Accordingly, for example, the amount of expansion and contraction in the radial direction when the voltage is supplied can be made larger. Specifically, in a mode where the second piezoelectric part is arranged split up into parts so as to surround the first piezoelectric part 15 (see later explained FIG. 4A), the plurality of split parts do not directly exert stresses upon each other. On the other hand, in the present embodiment, the plurality of parts configuring the second piezoelectric part 17 exert stresses upon each other in the circumferential direction. Accordingly, the second piezoelectric part 17 easily deforms in the radial direction due to the Poisson effect. As a result, for example, the transmission strength is improved. Also, the reception sensitivity is improved in the same way.

In the present embodiment, the first piezoelectric part 15 includes s part positioned at the center of the vibration part 5a, and the second piezoelectric part 17 includes a part positioned on the outer side of the vibration part 5a from the first piezoelectric part 15 and is larger in area overlaying the vibration part 5a than the area outside of it.

Accordingly, compared with the mode in which the area of the part positioned outside of the vibration part 5a is larger than the area overlaying the vibration part 5a (see later explained FIG. 4B), the second piezoelectric part 17 more easily directly influences the vibration of the vibration part 5a. As a result, for example, by suitably setting the material of the second piezoelectric part 17, the effect of improvement of the transmission strength and/or reception sensitivity becomes more remarkable.

(Modification)

In the following description, various modifications will be explained. Note that, in the following explanation, basically portions different from those in the embodiment will be explained. The matters which are not particularly referred to are the same as those in the embodiment. Further, in the following description, even if the shapes etc. of the members are different from those in the embodiment, for convenience, sometimes the same notations as those in the embodiment will be used.

(Modification Relating to Planar Shape of Piezoelectric Layer)

FIG. 4A is a plan view showing the configuration of a sensor 201 according to a modification and corresponds to FIG. 1B.

The sensor 201 is different from the embodiment in the planar shape of the piezoelectric layer and the planar shape of an upper electrode layer 211. Note that, the planar shape of the piezoelectric layer is the same as the planar shape of the upper electrode layer 211. In FIG. 4A, the piezoelectric layer is hidden behind the upper electrode layer 211, so is not shown. For convenience, in the explanation of FIG. 4A, notations in the embodiment will be used as the notations of the piezoelectric layer. The same is true for the explanation of FIG. 4B which will be given later.

In the sensor 201, the planar shape of the first piezoelectric part 15 (first electrode part 19) is the same as that in the embodiment. Further, the second piezoelectric part 17 (second electrode part 221) has a shape surrounding the first piezoelectric part 15 in the same way as the embodiment. However, the second piezoelectric part 17 according to the present modification, unlike the embodiment, has a plurality of split piezoelectric parts (split electrode parts 222) which are arranged so as to surround the first piezoelectric part 15. The number of the plurality of split piezoelectric parts may be suitably set. Further, the shapes and sizes of the plurality of split piezoelectric parts may be the same as each other or may be different from each other.

More specifically, in the example shown, the planar shape of the second piezoelectric part 17 (second electrode part 221) is the shape of the second piezoelectric part 17 (second electrode part 21) in the embodiment from which parts are removed at equal intervals. Accordingly, the plurality of split piezoelectric parts (split electrode parts 222) have the same shapes as each other. Further, these are substantially arc shapes (from another viewpoint, fan shapes with inner sides cut by concentric circles). Further, each of the split piezoelectric parts (split electrode parts 222) is larger in the area overlaying the cavity 13 (vibration part 5a) than the area on the outer side of the same (substantially 0 in the example shown).

The plurality of split electrode parts 222 are for example rendered the same potentials as each other. In the example shown, the plurality of split electrode parts 222 are connected to each other by a second connection conductor 225. The connection conductor 225 is for example the same as the second connection conductor 25 in the embodiment except for the planar shape.

With such a planar shape of the second piezoelectric part 17, for example, even if a region where the second piezoelectric part 17 is not arranged is formed at the periphery of the first piezoelectric part 15 in order to arrange the first connection conductor 23, the planar shape of the second piezoelectric part 17 can be made rotation symmetric by 360°/n (“n” is an integer of 2 or more). As a result, for example, the possibility of occurrence of unevenness in warping of the vibration part 5a can be reduced.

FIG. 4B is a plan view showing the configuration of a sensor 301 according to another modification and corresponds to FIG. 1B.

The sensor 301 differs from the embodiment in the planar shape of the piezoelectric layer 9 and the planar shape of an upper electrode layer 311. Specifically, in the sensor 301, in the second piezoelectric part 17 (second electrode part 321), the area overlaying the cavity 13 (vibration part 5a) (substantially 0 in the example shown) is smaller than the area outside of that.

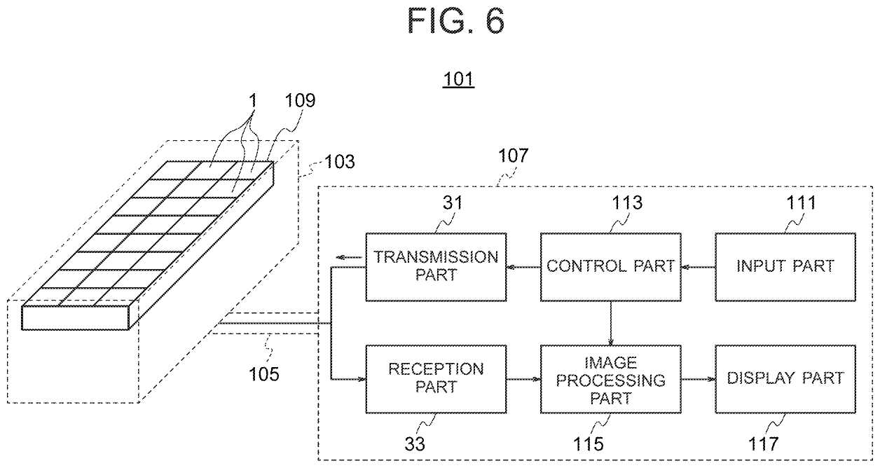

In another aspect, the second piezoelectric part 17, when viewed on a plane, does not overlay the vibration part 5a, but is adjacent to the vibration part 5a. The term “adjacent” referred to here for example means that a distance separating the vibration part 5a and the second piezoelectric part 17 is less than 10% (including 0%) of the maximum diameter (in a circle, it is the diameter) of the vibration part 5a when viewed on a plane. Further, for example, when a plurality of sensors are arranged (see the sensor 1 in FIG. 6), it means that the distance separating the vibration part 5a and the portion in the second piezoelectric part 17 which is closest to the vibration part 5a when viewed on a plane is shorter than the distance separating the closest portion and the other vibration part 5a.

Note that, the specific shape of the second piezoelectric part 17 (second electrode part 321), for example, in the same way as the embodiment, is an arc extending over a range of a semicircle or more (further 270° or more) with substantially a constant width so as to surround the first piezoelectric part 15 (first electrode part 19). The specific shape, unlike that shown, may be a shape of split in parts so as to surround the first piezoelectric part 15 in the same way as the modification in FIG. 4A.

In this way, even with a configuration where the second piezoelectric part 17 is adjacent to the vibration part 5a when viewed on a plane, the same effects as those by the embodiment are exhibited. For example, due to the second piezoelectric part 17 expanding or contracting in the planar direction, a compressive stress or tensile stress can be given to the outer peripheral part and upper surface of the vibration part 5a and the vibration part 5a can be made to flexurally deform at the outer peripheral part.

(Modification Relating to Thickness of Piezoelectric Layer)

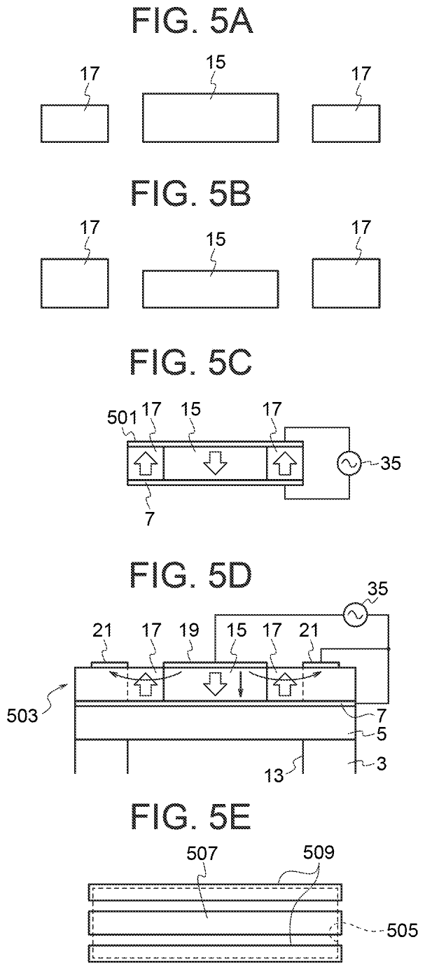

FIG. 5A is a schematic cross-sectional view (hatching showing the cross-section is omitted) showing a modification relating to the thicknesses of the first piezoelectric part 15 and second piezoelectric part 17.

As shown in this view, the first piezoelectric part 15 may be made thicker compared with the second piezoelectric part 17 as well. The degree of difference of thicknesses may be suitably set. For example, the first piezoelectric part 15 may be made thicker by 10% or more compared with the second piezoelectric part 17.

In such a configuration, for example, the first piezoelectric part 15 can more easily generate a large force due to be made thicker. On the other hand, the first piezoelectric part 15 has a large influence on the deformation of the vibration part 5a. Accordingly, for example, it becomes easier to make the deformation of the vibration part 5a larger and in turn easier to improve the transmission strength.

Therefore, for example, in a case where the transmission strength is considered to be more important than the reception sensitivity, the transmission strength can be improved in a pinpoint manner by forming the first piezoelectric part 15 larger in the “d” constant and thicker than the second piezoelectric part 17. Further, for example, when due to various circumstances the “d” constant of the first piezoelectric part 15 is smaller than that of the second piezoelectric part 17, the transmission strength can be secured by increasing the thickness of the first piezoelectric part 15.

FIG. 5B is a schematic cross-sectional view (hatching showing the cross-section is omitted) showing another modification relating to the thicknesses of the first piezoelectric part 15 and second piezoelectric part 17.

As shown in this view, conversely to FIG. 5A, the first piezoelectric part 15 may be made thinner compared with the second piezoelectric part 17 as well. The degree of difference of thicknesses may be suitably set. For example, the second piezoelectric part 17 may be thicker by 10% or more compared with the first piezoelectric part 15.

In such a configuration, for example, the vibration part 5a is apt to become larger in the amount of warping at the outer periphery side. As a result, for example, the displacement on the center side of the vibration part 5a due to warping on the outer periphery side of the vibration part 5a is apt to become larger. In this case, for example, compared with the case where the warping at the center side of the vibration part 5a is made larger to make the displacement at the center side of the vibration part 5a larger, the pressure wave becomes closer to a plane wave. As a result, for example, efficient transmission of ultrasound to the front of the sensor is facilitated.

Therefore, for example, by forming the second piezoelectric part 17 larger in the “d” constant and thicker compared with the first piezoelectric part 15, the above effect of the pressure wave becoming closer to a plane wave can be increased. Further, for example, when due to various circumstances the “d” constant of the second piezoelectric part 17 is smaller than that of the first piezoelectric part 15, the effect described above can be secured by forming the second piezoelectric part 17 thicker.

When summarizing FIG. 5A and FIG. 5B, the first piezoelectric part 15 and second piezoelectric part 17 may be made different in their thicknesses from each other. Any one of them may be made thicker. Also, the relationship between the relative magnitudes of the thicknesses and the type of material (relationship of magnitude of the “d” constant or “g” constant) may be suitably set. Note that, the structure making the thicknesses of the first piezoelectric part 15 and second piezoelectric part 17 different from each other may be applied to a sensor in which the materials for the first piezoelectric part 15 and second piezoelectric part 17 are the same as each other as well.

(Modification Relating to Planar Shape Etc. Of Upper Electrode Layer)

As shown in FIG. 5C, the first piezoelectric part 15 and the second piezoelectric part 17 may contact each other as well. Further, the upper electrode layer 501 may be provided in common for the first piezoelectric part 15 and second piezoelectric part 17 as well. Note that, as will be understood from the white arrows and driving part 35, the directions of polarization in this case and the configuration of the transmission part and the configuration of the reception part are for example the same as those in the example in FIG. 3B.

As shown in FIG. 5D, the second electrode part 21 need not be positioned on the second piezoelectric part 17 either. In this example, the second piezoelectric part 17 is adjacent to the first piezoelectric part 15. The direction of polarization is made inverse to the direction of polarization in the first piezoelectric part 15 as indicated by the white arrows. Further, the second electrode part 21 is positioned outside of the second piezoelectric part 17 when viewed on a plane. Further, the driving part 35 renders the lower electrode layer 7 and second electrode part 21 the same potential and supplies voltage to between them and the first electrode part 19.

In this case, in the first piezoelectric part 15, as indicated by the black arrow, in the same way as the embodiment, the voltage of the thickness direction is supplied and in turn the same deformation as the embodiment occurs. On the other hand, in the second piezoelectric part 17, as indicated by the black arrows, the voltage of planar direction is supplied. That is, voltage in a direction intersecting the polarization direction is supplied. As a result, the second piezoelectric part 17 undergoes so-called shear deformation. Due to this shear deformation, at the part of the vibration part 5a on the center side, displacement to the direction of warping by the deformation of the first piezoelectric part 15 is increased.

Note that, in this example, in the piezoelectric layer 503, the portion at the outer side from the second piezoelectric part 17 is sandwiched between the lower electrode layer 7 and the second electrode part 21 having the same potentials as each other, therefore basically does not deform. This portion need not be polarized either.

FIG. 5E is a schematic plan view of a sensor and corresponds to FIG. 1B. In the same way as FIG. 1B, the first piezoelectric part and the second piezoelectric part are the same in shapes as the first electrode part 507 and second electrode part 509 and are hidden behind these electrode parts, therefore are not shown.

As shown in this view, the cavity 505 (vibration part), first piezoelectric part (first electrode part 507), and second piezoelectric part (second electrode part 509) are not limited to circular shapes. Further, the first piezoelectric part (first electrode part 507) need not be positioned at the center of the cavity 13 in its entirety either. The second piezoelectric part (second electrode part 509) need not surround the first piezoelectric part (first electrode part 507) either.

In the example shown, the cavity 505 (vibration part) is rectangle. The first piezoelectric part (first electrode part 507) is rectangle so as to extend along the length of the cavity 505 at the center of the width of the cavity 505. The second piezoelectric part (second electrode part 509) is rectangle so as to extend along the length of the cavity 505 on the two sides of the width direction of the cavity 505. Note that, the second piezoelectric part (second electrode part 509), as explained with reference to FIG. 4B etc., may overlay or may not overlay the cavity 505.

(Example of Application)

FIG. 6 is a block diagram schematically showing the configuration of an ultrasonic diagnosis device 101 as an example of application of the sensor 1.

The ultrasonic diagnosis device 101 is provided with for example a probe 103 which is made to abut against a patient, a flexible cable 105 which is connected to the probe 103, and a device body 107 which is connected through the cable 105 to the probe 103.

The probe 103 for example has a sensor substrate 109. The sensor substrate 109 has a plurality of sensors 1 (may be sensors according to a modification as well) which are arranged in the planar direction (D1 axis direction and/or D2 axis direction).

Note that, although not particularly shown, a plurality of sensors 1 may be simultaneously formed by processing a wafer forming the base body 3. The base body 3 may be integrally formed over the plurality of sensors 1 as well. The lower electrode layer 7 may be formed in common for the plurality of sensors 1 (for example in a solid pattern). The second piezoelectric parts 17 and/or the second electrode parts 21 may be linked to each other between the sensors 1 which neighbor each other.

The plurality of sensors 1 may be ones receiving as inputs the same electrical signals as each other or may be ones receiving as inputs electrical signals which are different from each other (for example electrical signals for electronic scanning which are somewhat offset in phases). In the latter case, for example a common potential (for example reference potential) may be given to the lower electrode layers 7 among the plurality of sensors 1 while different potentials may be given to the upper electrode layers 11 (first electrode parts 19 and second electrode parts 21) among the plurality of sensors 1.

The device body 107 for example has an input part 111 accepting an operation of the user (for example a doctor or sonographer) and a control part 113 which controls the transmission part 31 based on a signal from the input part 111. Note that, the transmission part 31 is as already explained. Further, the device body 107 is provided with an image processing part 115 which performs image processing based on the signal from the already explained reception part 33 and the signal from the control part 113 and with a display part 117 which displays the image based on the signal from the image processing part 115.

By provision of the configuration as described above, the ultrasonic diagnosis device 101 can display a tomographic image of a patient in the display part 117. Note that, parts (for example amplifiers) of the transmission part 31 and reception part 33 may be provided in the probe 103 as well.

The present invention is not limited to the above embodiment and may be executed in various ways.

The planar shape of the piezoelectric layer is not limited to a shape in which the first piezoelectric part is positioned at the center side of the cavity (vibration part) and the second piezoelectric part is positioned outside of the same. For example, when viewed on a plane, the vibration part may be equally divided into two by a straight line passing through the vicinity of the center of the gravity of the figure, the first piezoelectric part may be positioned in one of the two parts, and the second piezoelectric part may be positioned in the other. In this case as well, for example, by configuring the first piezoelectric part by a material suitable for transmission and configuring the second piezoelectric part by a material suitable for reception, the balance between the transmission strength and the reception sensitivity can be made a suitable one. However, if, as in the embodiment, dividing it between the center side and the outer side of the vibration part, symmetry of deformation of the vibration part is more easily secured, and the materials can be made different from each other between regions having different modes of deformation.

Various planar shapes other than those illustrated are possible for the cavity (vibration part), first piezoelectric part, and second piezoelectric part. For example, they may be elliptical shapes or may be polygons other than rectangles. The electrode layer and piezoelectric layer were superposed on the membrane on the opposite side to the cavity. However, it is also possible to superpose them on the cavity side.

In the embodiment, as the ultrasonic sensor, a unimorph type was illustrated. However, it may be a bimorph type as well. That is, a piezoelectric layer which is inverse in the direction of polarization to the piezoelectric layer 9 may be arranged between the piezoelectric layer 9 and the lower electrode layer 7 or between the piezoelectric layer 9 and the upper electrode layer 11. In this case, the membrane 5 is unnecessary. Further, in the piezoelectric layer 9, when the direction of polarization is different for each region (between the first piezoelectric part and the second piezoelectric part), the piezoelectric layer superposed on the piezoelectric layer 9 may also be different in the direction of polarization for each region. Further, the piezoelectric layer superposed on the piezoelectric layer 9 may be configured in its entirety by the same material. Alternatively, in the same way as the piezoelectric layer 9, it may be configured by a different material for each region.

In the embodiment, voltages were supplied to both of the first piezoelectric part and second piezoelectric part, and electrical signals were extracted from both of the first piezoelectric part and second piezoelectric part. However, the voltage may be supplied to only one of the first piezoelectric part and second piezoelectric part (for example the piezoelectric part configured by a material which is relatively more suitable for oscillation) and/or the electrical signal may be extracted from only the other of the first piezoelectric part and second piezoelectric part (for example the piezoelectric part configured by a material which is relatively more suitable for reception). According to such an aspect, for example, improvement of the transmission strength and/or reception sensitivity can be expected.

### REFERENCE SIGNS LIST

1 . . . sensor (ultrasonic sensor), 5 . . . membrane, 5a . . . vibration part, 7 . . . lower electrode layer, 9 . . . piezoelectric layer, 11 . . . upper electrode layer, 15 . . . first piezoelectric part, and 17 . . . second piezoelectric part.

1. An ultrasonic sensor comprising
a lower electrode layer facing a cavity,
a piezoelectric layer located on the lower electrode layer, and
an upper electrode layer located on the piezoelectric layer, wherein
the piezoelectric layer comprises
a first piezoelectric part
which comprises a first material having piezoelectricity and
which at least partially overlays the cavity when viewed on a plane and
a second piezoelectric part
which comprises a second material having piezoelectricity and being different in at least one of a “g” constant and “d” constant from the first material and
which, when viewed on a plane, is located in a region different from an arrangement region of the first piezoelectric part, and at least partially overlays or is adjacent to the cavity.

2. The ultrasonic sensor according to claim 1, wherein one of the first material and the second material is larger in the “d” constant and smaller in the “g” constant relative to the other.

3. The ultrasonic sensor according to claim 1, wherein:
the first piezoelectric part comprises a portion which is located at a center of the cavity when viewed on a plane, and
the second piezoelectric part comprises a portion which is located at an outer side of the cavity from the first piezoelectric part when viewed on a plane.

4. The ultrasonic sensor according to claim 3, wherein:
the first material is larger in the “d” constant than the second material, and
the second material is larger in the “g” constant than the first material.

5. The ultrasonic sensor according to claim 3, wherein:
the first material is larger in the “g” constant than the second material, and
the second material is larger in the “d” constant than the first material.

6. The ultrasonic sensor according to claim 3, wherein the second piezoelectric part extends over a range exceeding a semicircle so as to surround the first piezoelectric part when viewed on a plane.

7. The ultrasonic sensor according to claim 3, wherein, in the second piezoelectric part, an area overlaying the cavity is larger than an area an outside of that.

8. The ultrasonic sensor according to claim 1, wherein a thickness of the first piezoelectric part and a thickness of the second piezoelectric part are different from each other.
